(19) United States
(12) Patent Application Publication
(10) Pub. No.: US 2021/0054319 A1
(43) Pub. Date: Feb. 25, 2021

(54) FLOW BIOREACTOR DEVICE FOR MONITORING CELLULAR DYNAMICS

(71) Applicant: Technion Research & Development Foundation Limited, Haifa (IL)

(72) Inventors: Shulamit LEVENBERG, Moreshet (IL); Barak ZOHAR, Karmiel (IL)

(73) Assignee: Technion Research & Development Foundation Limited, Haifa (IL)

(21) Appl. No.: 17/045,763

(22) PCT Filed: Apr. 10, 2019
(86) PCT No.: PCT/IL2019/050404

§ 371 Date: Oct. 7, 2020

Related U.S. Application Data
(60) Provisional application No. 62/656,355, filed on Apr. 12, 2018.

(51) Int. Cl. C12M 3/00; C12M 1/00

(52) U.S. Cl. CPC C12M 21/08; C12M 23/22; C12M 29/10

(57) ABSTRACT

A flow bioreactor device for monitoring cellular dynamics may include a housing having one or a plurality of conduits to place one or a plurality of tissue samples inside each of said one or a plurality of conduits, wherein each of said one or a plurality of conduits has an inlet for introducing a flow of a liquid in a direction of flow through that conduit, wherein said one or a plurality of conduits are fluidically linked to an outlet for discharging the liquid, and wherein at least a portion of the housing is transparent so as to provide a line of sight for viewing or for an optical device along an elongated axis of any of said one or a plurality of conduits, the line of sight being substantially parallel to the direction of flow through that conduit.

### FIELD OF THE INVENTION

The present invention relates to the field of tissue engineering and, more particularly, to a flow bioreactor device, having one or a plurality of channels, for monitoring cellular dynamics under various flow conditions.

### BACKGROUND OF THE INVENTION

Tissue engineering techniques typically make use of constructs, which generally involve three-dimensional (3D) polymeric scaffolds in combination with one or more cells types, to form implantable tissue-like devices for replacing damaged tissue. Additionally, engineered tissues can serve as models in in-vitro study of normal and diseased biological processes at the tissue level. Vascularization of engineered tissue constructs in-vitro is a challenge of great significance for regenerative medicine. Without a stable and perfusable blood vessel network to provide oxygen and nutrients, cells cannot survive once the tissue dimensions grow beyond several hundred microns due to diffusive limitations. Thus, perfusion bioreactors play an important role in creating the specific culture conditions necessary for the development of 3D engineered tissues by improving nutrient transportation and waste removal and by generating mechanical stimulation in the form of shear stress.

Direct perfusion culture has been shown to be beneficial for culturing various types of engineered tissues such as cardiac, hepatic, cartilage and bone tissue, with even very low flow rates inducing widespread changes in gene and protein expression in multiple cell types. Although 3D culturing under dynamic conditions better mimics natural tissue environments, the vast majority of in vitro research is still conducted under static conditions, largely due to the ease of handling and low risk for contamination. In addition, microfluidic devices serving as perfusion platforms are still limited in geometry and scale, involve complicated fabrication techniques, and require designated facilities. Thus, direct flow bioreactors are currently the ultimate systems for cultivating clinically relevant engineered tissues but are unsuitable for in situ imaging studies.

### SUMMARY OF THE INVENTION

There is provided, according to some embodiments of the present invention, a flow bioreactor device for monitoring cellular dynamics. The device may include a housing with one or a plurality of conduits to place one or a plurality of tissue samples inside each of said one or a plurality of conduits. Each of the conduits may have an inlet for introducing a flow of a liquid in a direction of flow through that conduit. The conduits may be fluidically linked to an outlet for discharging the liquid. At least a portion of the housing may be transparent so as to provide a line of sight for viewing or for an optical device along an elongated axis of any of the conduits, the line of sight being substantially parallel to the direction of flow through that conduit.

In some embodiments of the present invention, the device comprises a housing formed from detachable parts.

In some embodiments of the present invention, the housing comprises a main body and a cover.

In some embodiments of the present invention, said one or a plurality of conduits are located in the main body.

In some embodiments of the present invention, said one or a plurality of conduits include each an upright portion.

In some embodiments of the present invention, the inlet of each of said one or a plurality of conduits is located on the main body.

In some embodiments of the present invention, each of said one or a plurality of conduits has an opening on a top surface of the main body.

In some embodiments of the present invention, the cover has a bottom surface designed to face the top surface of the main body.

In some embodiments of the present invention, the device includes one or a plurality of washers for sealing one or a plurality of links between said one or a plurality of conduits and an internal chamber within the cover.

In some embodiments of the present invention, the cover comprises a hollow body defining an internal chamber.

In some embodiments of the present invention, the cover comprises a transparent window which, when the cover is mounted over the main body, facilitates a line of sight through the window that is aligned with said one or a plurality of conduits.

In some embodiments of the present invention, the window is made of glass.

In some embodiments of the present invention, the window comprises a glass disk, which is secured in position by a holder.

In some embodiments of the present invention, an O-ring is provided to seal the window.

In some embodiments of the present invention, the main body and the cover are secured together by a screw.

In some embodiments of the present invention, the device is made from one or more materials selected from the group of materials consisting of poly-methyl methacrylate (PMMA) and poly-ether ketone (PEEK).

In some embodiments of the present invention, the device is fluidically connected to a perfusion system.

In some embodiments of the present invention, the perfusion system is a closed-loop system.

In some embodiments of the present invention, the perfusion system comprises one or a plurality of pumps, each of said one or a plurality of pumps separately fluidically connected to an inlet of one of said one or a plurality of conduits.

In some embodiments of the present invention, said one or a plurality of pumps comprises one or a plurality of peristaltic pumps.

### BRIEF DESCRIPTION OF THE DRAWINGS

In order for the present invention to be better understood and for its practical applications to be appreciated, the following figures are provided and referenced hereafter. It should be noted that the figures are given as examples only and in no way limit the scope of the invention. Like components are denoted by like reference numerals.

FIG. 1 is an exploded view of a multi-channel flow bioreactor device, according to some embodiments of the present invention.

FIG. 2 shows the two main parts of a multi-channel flow bioreactor device, according to some embodiments of the present invention, in a disassembled state.

FIG. 3 illustrates an exemplary set-up for use with a flow bioreactor device, according to some embodiments of the present invention.

FIG. 4 shows positioning of a multi-channel flow bioreactor device in front of an objective of a microscope, according to some embodiments of the invention.

### DETAILED DESCRIPTION OF THE INVENTION

In the following detailed description, numerous specific details are set forth in order to provide a thorough understanding of the methods and systems. However, it will be understood by those skilled in the art that the present methods and systems may be practiced without these specific details. In other instances, well-known methods, procedures, and components have not been described in detail so as not to obscure the present methods and systems.

Although the examples disclosed and discussed herein are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method examples described herein are not constrained to a particular order or sequence. Additionally, some of the described method examples or elements thereof can occur or be performed at the same point in time.

Unless specifically stated otherwise, as apparent from the following discussions, it is appreciated that throughout the specification, discussions utilizing terms such as “adding”, “associating” “selecting,” “evaluating,” “processing,” “computing,” “calculating,” “determining,” “designating,” “allocating” or the like, refer to the actions and/or processes of a computer, computer processor or computing system, or similar electronic computing device, that manipulate, execute and/or transform data represented as physical, such as electronic, quantities within the computing system's registers and/or memories into other data similarly represented as physical quantities within the computing system's memories, registers or other such information storage, transmission or display devices.

According to some embodiments of the present invention, there is provided a flow bioreactor device, having one or a plurality of channels, which facilitates real-time imaging of cellular dynamics in 3D constructs under various controlled flow conditions in the said one or a plurality of channels (hereinafter, “channels” refers to either a single channel or a plurality of channels, unless stated otherwise).

In some embodiments of the present invention, the bioreactor device is designed to be loaded with one or a plurality (e.g., 2-4) replicated 3D engineered constructs for long cultivation periods under diverse controlled flow stimulations. Viewing into and along the channels is made possible by providing a clear field of view into and along these channels. The bioreactor device may be designed to be compatible with an imaging device (e.g., an upright confocal microscope) for 3D scanning, which can be used to monitor cell dynamics.

As an example, a flow bioreactor device according to some embodiments may be used for monitoring angiogenic processes in 3D engineered tissue cultured under direct flow conditions. Such platform allows defining the kinetics of microvascular formation in 3D engineered tissue, as determined by total vessel length and the impact of flow on vascularization rate, as indicated by total vessel elongation and tip cell dynamics. In addition, the vascular network topology may be characterized by tracking the structural angles during vascular network development.

According to some embodiments, a flow bioreactor device facilitates cross-sectional views for exploring the dynamics of endothelial and supporting cell motility, proliferation and arrangement under physiological wall shear stress stimuli applied by flow through a channel in a 3D fused construct, and a tissue engineered vessel graft (TEVG). A flow bioreactor device, according to some embodiments of the invention, may be a useful platform for easily and conveniently quantifying and analyzing cellular dynamics in 3D engineered tissues cultured under controlled flow conditions.

According to some embodiments of the present invention, a flow bioreactor device includes one or a plurality of channels into which a 3D biological construct may be inserted, and through which liquid may flow, so as to subject the 3D biological construct to a predetermined, controlled and programed flow conditions. Flow conditions may include, for example, varying materials (e.g., cells, supply of nutrients, waste removal and dissolved gas supply, etc.), temperatures, pressure, mechanical stimulation (shear stress), etc. According to some embodiments of the present invention, a clear field of view is provided into each of the channels, substantially parallel to the direction of the flow through that channel, so as to allow viewing (e.g., by a human eye, or by an optical device) the content of the channels, and allow focusing imaging devices onto a desired point across a lateral cross-section of the channel, and within desired depth (depending on the level of opacity of the materials inside the channels).

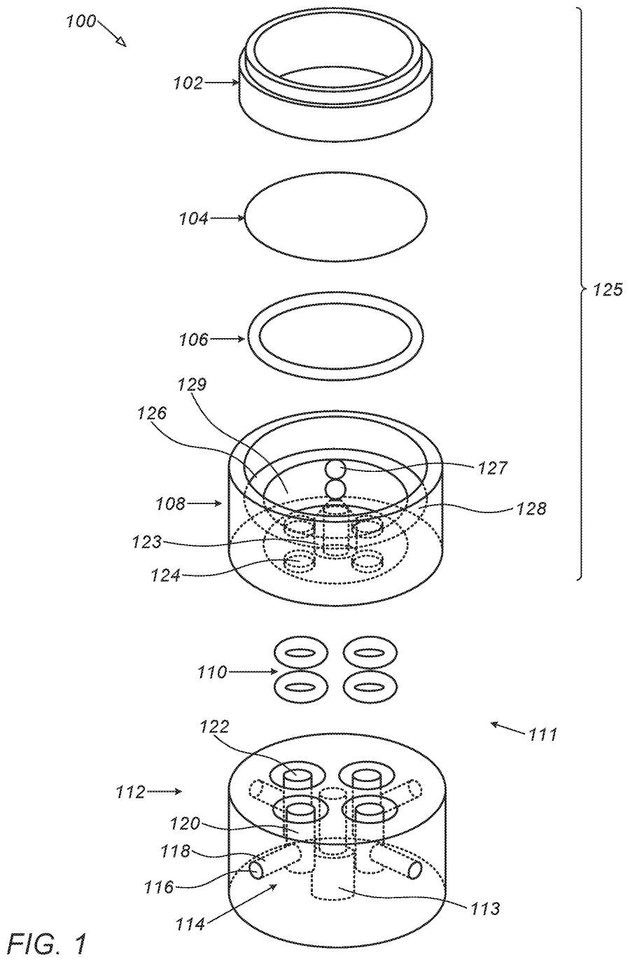

FIG. 1 is an exploded view of a multi-channel flow bioreactor device, according to some embodiments of the present invention.

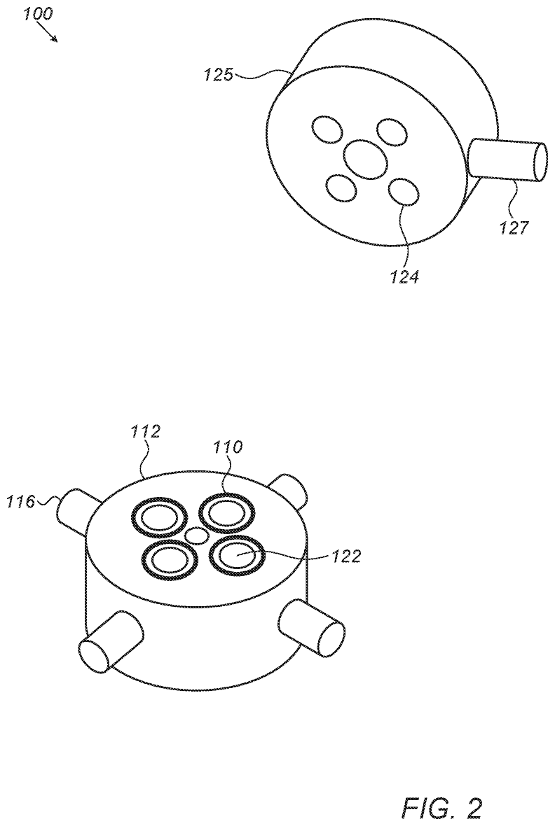

FIG. 2 shows the two main parts of a multi-channel flow bioreactor device, according to some embodiments of the present invention, in a disassembled state.

Multi-channel flow bioreactor device 100 includes a housing 111 that may include a main body 112 and cover 125. Main body 112, which is shown for example in a cylindrical shape, includes one or a plurality of conduits 114 (in the example of FIG. 1, there are four conduits). Conduits 114 may include main upright portions 120 that pass substantially parallel to each other within the main body 112. Each conduit 114 may be provided with an inlet 116 for separately introducing a liquid into that conduit (e.g., via passage 118), and an outlet 122 on a top surface of the cylindrical main body 112 through which the flow passing through the channel may exit. In some embodiments of the present invention, the supply line into each of inlets 116 of the main body 112 may include a separate pump, in order to independently control the flow conditions for each of the conduits.

A cover 125 may be mounted over main body 112.

Cover 125 may include a hollow cover body 108 defining an internal chamber 129 with a top opening which may be covered by a window 104. Window 104 may comprise a removable transparent disk that may rest over a shoulder 128 annularly along a periphery of the top opening, over which holder 102 may be screwed (or otherwise securely mounted), to hold window 104 in place. An O-ring 106 may be used to seal window 104 of the cover. Each of outlets 122 of main body 112 is designed to be coupled to one of inlets 124 of cover 125, which are located on a bottom surface of cover 125 designed to face the top surface of main body 112, when cover 125 is properly mounted over main body 112. Washers 110 may be provided to prevent leakage. Window 104 is designed to be aligned with the upright portions of conduits 114 so that a line of sight of a viewing or imaging device placed in front of the window 104 may align with the upright portion of at least one of the plurality of conduits, substantially parallel to the wall (if the conduits are tubular) or walls of that conduit.

A bore 113 in main body 112 and a corresponding bore 123 in cover 125 may be provided for inserting a screw to firmly hold both parts together.

Being constructed from two detachable main parts—main body 112 and cover 125—makes flow bioreactor device 100 suitable for aseptic handling as it makes it very easy to insert, replace and remove a tissue sample (e.g., tissue-growth scaffold, ex-vivo tissue piece, cell culture or multi-culture, of natural or engineered tissue) into a conduit of the flow bioreactor device 100 through the opening on the top surface of the main body, and then mount the cover onto the main body and fasten them together (e.g., using a screw for securing the parts together). When the use of the flow bioreactor is finished, the two parts may be disengaged to allow cleaning and otherwise preparing the device for another use.

In some embodiments of the present invention, the flow bioreactor is disposable, designed for a single use.

The main body and the cover of a flow bioreactor device, according to some embodiments of the present invention, may be made of an inert impervious material, such as, for example, poly-methyl methacrylate (PMMA) or poly-ether ketone (PEEK) (e.g., autoclavable version). Window 104 may be made of glass (e.g., 0.15 mm thickness of thermo scientific glass). Cover 125 may be provided with an outlet 127 through which liquid from within internal chamber 129 of cover 125 may be discharged.

Some, if not all, of the parts of the flow bioreactor device, according to some embodiments of the invention, may be made transparent to allow light (e.g., visible light, UV light, etc.) to traverse into and/or out of the conduits of the device, and in particular in the line of sight of a human eye or an optical device (e.g., a microscope, camera, optical sensors, such as, for example, IR sensor, spectroscope, etc.) used to view into the device from over its cover.

Typically, the objective of a microscope, may be placed over window 104, to provide a line of sight into one or more (depending on the chosen objective) of the upright parts 120 of conduits 114. According to some embodiments of the invention, the line of sight of the viewing into the conduit (in which a tissue sample may be placed) is substantially parallel to the direction of flow of the fluid through that conduit, e.g., substantially parallel to the longitudinal axis of the conduit, extending inside the conduit substantially parallel to the walls—or tubular wall in the case of tubular conduits.

According to some embodiments of the present invention, the design of the flow bioreactor device, and more specifically—the upright conduits in the main body and the internal chamber of the cover—ensures that gas bubbles are not trapped inside conduits 114. Any gas bubble that is found in conduits 114 is bound to float up into internal chamber 129 of cover 125, thereby getting out of the conduits 114 and out of view.

In some embodiments of the present invention, window 104 may be designed to be slightly slanted, so as to cause gas bubbles that float up to be moved aside. In some embodiments, the slanted window 104 may be designed to drive the gas bubbles in the direction of outlet 127, so as to encourage the bubbles to exit the device.

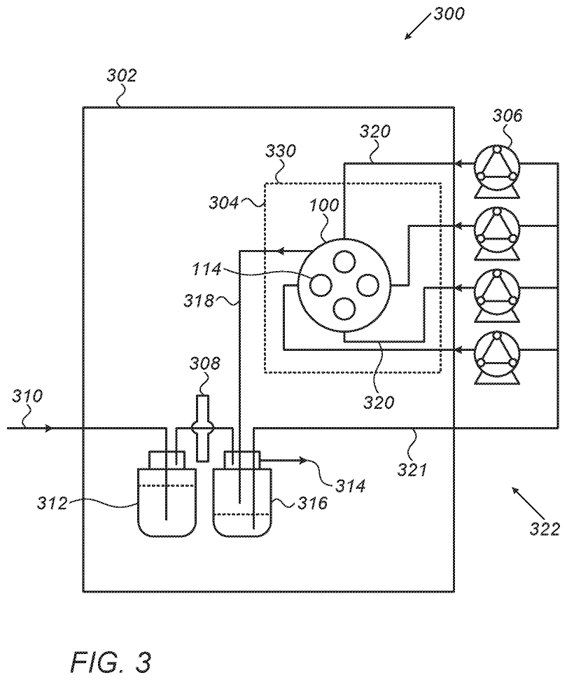

FIG. 3 illustrates an exemplary set-up 300 for use with a flow bioreactor device 100, according to some embodiments of the present invention.

Multi-channel flow bioreactor device 100 may be loaded with a plurality of tissue samples, each tissue sample placed in a separate channel (conduits 114). A perfusion system 322 may be provided to perfuse the tissue samples. The tissue samples may be perfused separately, by connecting the inlet (see 116 in FIG. 1) of each of the conduits 114 of the flow bioreactor device 100 to a separate supply channel 320, each supply channel powered by a designated pump 306. The separate inlets allow separate supply and separate control for each inlet, so that the changes in the mass-flow rate (or other flow condition/s) in one conduit that may occur do not necessarily affect the mass-flow rate (or other flow condition/s) in the other conduits. The pumps are all fluidically via main supply channel 321 linked to perfusion medium reservoir 316, which supplies the medium to be perfused onto the tissue samples. A humidifier 312 may be provided that is designed to mix air entered via air-vent 310 into the humidifier 312 and water contained in the humidifier, and supply the air-water mixture, via an air-filter 308 into the medium reservoir 316. In some embodiments of the present invention, pumps 306 are peristaltic pumps.

The perfusion medium is perfused onto the tissue samples in each of the conduits and then discharged through the internal chamber of the cover out of the outlet (see 127, in FIG. 1) of the bioreactor device 100, via collection channel 318, to be collected into medium reservoir 316, for reuse.

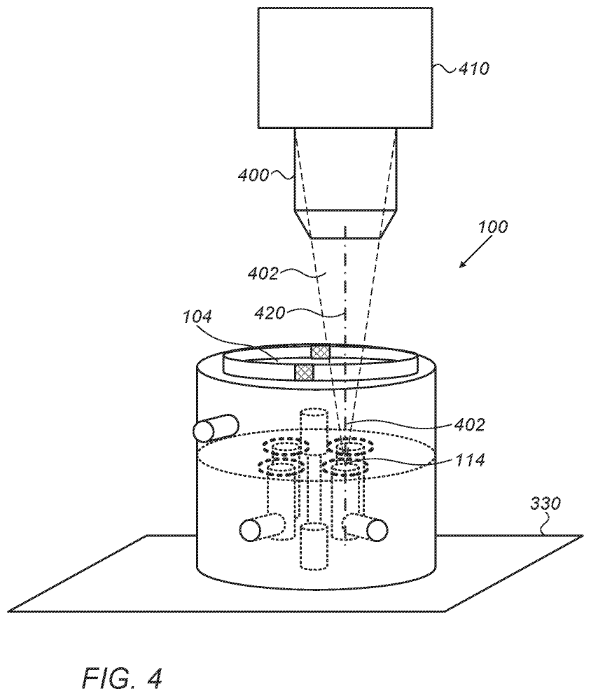

The flow bioreactor device 100 may be placed inside an upright confocal microscope chamber 302, which may be temperature controlled. An X-Y movable table 330 may be provided, on which one or a plurality of flow bioreactor devices according to some embodiments of the invention may be mounted (e.g., fixed to). The X-Y table 330 allows repositioning of the flow bioreactor device (or devices) along any of the two orthogonal axes (X, Y, and in some embodiments of the present invention also along the Z axis) so that a desired conduit may be placed in front of the objective 400 of the microscope 410, as shown in FIG. 4, to allow focusing 402 of the microscope along the line of sight 420 of the microscope 410. Having window 104 over the upright portion of conduit 114 of the flow bioreactor device 100 facilitates both scanning across the width of the conduit 114 and/or focusing deeper or shallower along the line of sight 420. The quality of the viewing and images obtained may typically depend on the opacity level of the liquid inside the conduit and depth of the focusing. Typically, in tissue-growth viewing and imaging, fluorescence is employed (coloring of cells in fluorescent agents).

It is noted that other perfusion systems may also be used, not necessarily closed-loop. However, a closed-loop system may have its advantages.

Hereinafter, an experimental example of the use of a flow bioreactor device, according to some embodiments of the invention is presented.

### EXPERIMENTAL EXAMPLE

Materials and Methods Used:

Cell Culture.

Zs-green-expressing human adipose microvascular endothelial cells (HAMECs) (Passage 5-7, ScienceCell) and Zs-green-expressing human umbilical vein endothelial cells (HUVECs; Lonza) were cultured in tissue culture flasks. HAMECs were cultivated in endothelial cell medium (ScienceCell), supplemented with 5% fetal bovine serums (FBS) (ScienceCell) and endothelial cell growth supplement (ScienceCell) and HUVECs were cultivated in EGM-2 medium, supplemented with a bullet kit containing FBS, hydrocortisone, hFGF-β, VEGF, R3-IGF-1, hEGF, GA-1000 and heparin (Lonza). Neonatal human dermal fibroblasts (HNDFs) (Passage 6-8, Lonza) were cultured on tissue culture flasks in DMEM (Gibco) supplemented with 10% fetal bovine serum (FBS) (Hyclone), 1% non-essential amino acids, 0.2% β-mercaptoethanol and 1% penstrep (Sigma Aldrich).

Vascular 3D Porous Construct Preparation.

Macroporous poly-L-lactic acid (PLLA) (Polysciences, Warrington) and poly-L-glycolic acid (PLGA) (Boehringer Ingelhein) (weight ratio of 1:1) scaffolds were prepared using a porogen leaching protocol as previously described and finally cut to a final disc-shaped construct (diameter of 6 mm and thickness of 1 mm). Endothelial Cells (EC) and HNDFs were mixed (500,000 and 100,000 cell, respectively, per scaffold) in 14 μL human fibrin gel, prepared from a mixture (volume ratio of 1:1) of thrombin solution (15 mg/mL, Johnson & Johnson Medical, Israel) and human fibrinogen solution (5 mU/mL, Johnson & Johnson Medical), and then immediately seeded upon the macroporous scaffolds. Scaffolds were then incubated (30 min, 37° C., 5% CO₂) on a 12-well non-tissue culture plate. Co-culture medium (a 1:1 mixture of the two respective cell media) was added (1-3 mL per scaffold) and replaced every 2-3 days. Scaffolds were cultured under static conditions (37° C., 5% CO₂) for-35 days before being loaded in the MFV bioreactor.

Fused Macrochannel Construct Preparation.

HNDFs (100,000 cell per scaffold) and HAMECs (500,000 cell per scaffold) were seeded separately in 3D monoculture constructs, as was described above. After 2 days of culture under static conditions, constructs embedded with HAMECs were cut into ring shapes (outer diameter of 2 mm and inner diameter of 1 mm, cut using a biopsy punch) and fused into constructs embedded with HNDFs that were cultured for 5 days under static conditions.

Tissue Engineered Vessel Graft (TEVG) Preparation.

Tubular porous scaffolds were prepared according to a modified previously described protocol. Briefly, a 5% (wt/vol) solution of PLLA (Polysciences Inc.) in chloroform was prepared. NaCl particles were ground and sieved with a 125 μm pore sieve. 1 ml of sieved NaCl particles was mixed into 5 ml of the PLLA solution. A 2.5 mm diameter stainless steel rod was dipped into the NaC:PLLA mixture for 10 seconds, then removed from the solution and air-dried at room temperature for 2 minutes. This formed a NaC:PLLA layer surrounding the metal rod. The rod was then immersed in methanol for 30 seconds to facilitate the separation of the overlaying NaCl:PLLA layer from the rod. The tubular NaC:PLLA layer was removed and placed in distilled water over night to remove the NaCl particles. Scaffolds were then dried at room temperature for 2 hours and cut to a length of 10 mm. Porous scaffolds were immersed in ethanol for 30 minutes for sterilization. The inner surface of the tubular scaffolds was coated using a 50 μg/ml human fibronectin solution (Sigma) for 1 hour at 37° C. After the remaining fibronectin solution was rinsed with PBS, 15 μl of HAMEC suspension with a concentration of 5-10⁶ cell/ml was seeded into the lumen of the scaffold. To achieve a uniform cell lining, the scaffolds were put at 37° C. for 2 hours under axial rotation. The non-attached cells were washed out with HAMECs medium. Tubular scaffolds were cultured under static conditions (37° C., 5% CO₂) for 2 days and then placed through a silicon perforated cup-like holder filled with 5·10⁴ HNDFs suspended in fibrin (FIG. 9A). Constructs were cultured (Co-culture medium) for an additional day under static conditions before been loaded in the multi-channel flow bioreactor device.

Multi-Channel Flow Bioreactor Device.

The multi-channel flow bioreactor device was designed to be loaded with four scaffolds placed in separated flow conduits. Each chamber is connected to a closed loop perfusion system (e.g., 322 in FIG. 3), designed to be placed inside standard upright confocal microscope chamber (e.g., 302, FIG. 3). The tubing (e.g., 318, 320, 321 in FIG. 3) used to connect the system was made of a thermoplastic elastomer (ID0.031×OD0.094, C-FLEX®, Cole-Parmer) and the tubing for the peristaltic pump (e.g., 306 in FIG. 3) was made of PharMed® BPT 1.14 mm ID RED/RED, ISMATEC)). These tubings were chosen because they are both autoclavable and have low CO₂ and O₂ permeability. The bioreactor device was placed inside the temperature-controlled chamber (37° C.) of the microscope. To control physiological pH levels (7.2-7.4) inside the conduits and internal chamber of the bioreactor device and avoid medium evaporation, air with 8% CO₂ was bubbled by a sintered sparger through warm ultra-pure water. The gas mixture was filtered and supplied directly to the medium reservoir in overlay gassing mode.

Flow Bioreactor Device Sterilization and Handling.

Several multi-channel bioreactors were used. Each bioreactor device was washed with purified water and PBS followed by sterilization in an autoclave (121° C. for 30 min). When using the PMMA chamber, it was disinfected by soaking in 70% volume fraction ethanol for 0.5 h and rinsed twice with PBS. The chamber was further assembled and aseptically connected to the sterile perfusion system in a laminar flow hood. To test the system integrity, the multi-channel flow bioreactor was incubated with the relevant culture medium, which was circulated for at least 24 hours before scaffold loading. 3D scaffolds were placed aseptically (in a laminar flow hood) inside conduits of the multi-channel flow bioreactor device and 20 ml (5 ml per each scaffold) of relevant co-culture medium was poured into the medium reservoir bottle. Then, the flow bioreactors were placed inside the confocal chamber of an upright microscope, the reservoir bottles were connected to a gas supply and the flow bioreactor devices were fixed to a confocal robotic plate (X-Y movable table 330 in FIG. 3). During the cultivation period, the medium in the reservoir bottle was replaced every 72 hours.

Validation and Characterization of Culture Conditions.

During the cultivation period, culture medium temperature and pH were checked daily and maintained within the physiological range of 36-37.5° C. (Brannan mercury thermometer) and pH 7.2-7.4 (Mettler Toledo pH meter). In addition, culture medium volume was measured after 3 days to ensure negligible decrease in working volume caused by water evaporation. Flow velocity inside the MFV chamber was characterized by a computational fluid dynamics (CFD) model to ensure uniform flow distribution within the separated conduits. Steady state CFD simulations were performed in FLUENT using the pressure based coupled solver. The selected mesh was constructed in GAMBIT on the basis of the flow bioreactor device conduit CAD model and contained 1 million cells. The maximum Reynolds number estimated in the system was 4.5 so laminar flow was assumed. The maximum Damkohler number estimated in the system was 1.26×10⁻⁵ for oxygen consumption vs transportation rate. The very low Damkohler number indicated that there is no nutrient transportation limitation using our flow conditions. The results were post processed in the native Ansys® post processor. During cultivation time, a flow rate of 0.1 mL/min, corresponding to a mean shear stress of 0.75 dyne/cm², was set for the flow through each conduit separately. The same flow rate (0.1 mL/min) was also set for the fused macrochannel construct. Flow velocity and wall shear stress were characterized by a CFD model using the FLUENT software.

Image Acquisition and Processing.

For each scaffold, an initial position of the robotic X-Y table was set to the center mass of the scaffold for acquiring 2.5×2.5 mm (width of view) images using a Leica PLANAPO 2.0X/WD 39 mm objective. The z-stack was only defined after any medium replacement, to capture the maximum depth of field (400-600 μm, split into at least 30 z-stacks). The scaffolds were scanned in time lapse over 1 hour, by a LEICA SuperZoom Z6 confocal upright microscope. 4-D (XYZT) confocal z-stacks were converted to 2D time-series TIFF stacks by performing z-projections at each time step using the NIH ImageJ software. Then, images were processed by contrast enhancement (stack histogram equalization and normalization, 0.4% saturated pixels).

Image Analysis.

Each scaffold and frame were identically processed using the Angiotool® interface, or by applying the IMARIS fully automated FilamenTracer software (IMARIS 8.2.0) to quantify total vessel length. Total vessel elongation rate was defined as the slope of the total vessel length trend, calculated by performing linear regression. Vascular branching angle, calculated by IMARIS, was defined as the angle between the extending lines connecting the branch point with neighboring points. Vascular branch to branch/end point angles were defined as the angles between the extending lines connecting the branch point with the neighboring branch points and the terminal points. Vascular orientation angle was defined as the angle formed between extending line connecting distal vertices of the vessel segment and X-axis of image within XY plane. When measuring the branching angles, only the close vicinity of the junction was considered, and the origin of the parent artery and the destination of its branches were ignored. The mean angles were calculated as the mean of the average of 250-1000 angles segmented in each scaffold. Tip cell tracking was performed using the NIH ImageJ software manual tracking plug. Tracking was done “blindly” for 10 randomly selected tip cells in each scaffold.

Measurements were performed in triplicates, at minimum, and images were scanned, processed and analyzed using an identical setup. Means were plotted, with error bars representing the standard error of the mean (SEM). Statistical comparisons were performed using the Student's t test with a 95% confidence limit (two-tailed and unequal variance). Differences with a p-value<0.05 were considered statistically significant.

Results and Discussions.

The formation of vascular networks within 3D engineered tissue constructs has been described previously, following real-time imaging of endothelial cells cultured under static conditions. However, within the body, endothelial cells are also affected by biomechanical and biochemical stimulations induced by blood flow. By using a flow bioreactor, in accordance with some embodiments of the present invention, it was possible to visualize and quantify angiogenic processes as they form under flow-induced physiologically relevant shear stress. Scaffolds were 3D scanned inside the MFV bioreactor every hour from day 7 to 13 post-seeding, while the cells remained viable and sterile within 8 days of culture under physiological flow conditions. Moreover, GFP-labeled HUVECs formed de-novo micro-vascular networks that were clearly visible for 3D image acquisition via confocal microscope. A rapid increase in total vessel length was observed during days 7-9 post-seeding, followed by a stationary phase, which began on day 11. During days 7-9, total vessel length rose approximately linearly (R²˜0.95), at a constant elongation rate of ˜53.4 mm/day, which then gradually declined to zero after day 9. The dynamic of the total vessel length can be explained by the biological mechanism of angiogenesis process. Angiogenesis refers to the expansion of existing vascular networks into new blood vessels via several mechanisms such as sprouting, intussusception (vessel splitting) and/or vessel fusion. Previous studies have shown that the process requires several types of specialized, distinctly differentiated endothelial cells. These include “tip cells” which lead the way using filopodia, “stalk cells”, which remain behind the tip cells and maintain the stalk of the vessel, and the quiescent “phalanx cells”, which line new vessel branches once they are integrated into the network. Finally, tip cells anastomose with existing vessels and stop sprouting. Intuitively, the decreased total vessel elongation rate, during days 9-11, is affected by the increased probability of a tip cell to meet a pre-existing vessel, which increases as the total vessel length increases.

The flow bioreactor, according to some embodiments of the present invention, provides a novel culture platform that may be used in testing the effect of flow on cell activity in 3D cultures. To demonstrate its capacities, the effect of direct flow on the vascular elongation rate was tested during angiogenesis occurring in 3D constructs (n=4). Control samples included pre-vascularized constructs subjected to the same culture conditions, but without direct flow-stimulated shear stress. In these samples (n=3), the medium was only circulated through a nearby bypass channel. The mean of total vessel elongation rates measured as 1.1 mm/hr (R²˜0.92) and 0.46 mm/hr (R²˜0.91) for the flow and control conditions, respectively, amounting to a 70% increase (p<0.07) in total vessel elongation rate upon shear stimulation. These findings corroborate with an earlier study, in which it was demonstrated the significant impact of direct flow through 3D constructs on vessel formation, as manifested by a ˜100% increase in total vessel length after 2 days of culture under flow conditions, p<0.05. These findings suggest that the enhanced vascularization is likely to be explained by a change in the kinetics of the angiogenic process.

Topological analysis of the newly formed vascular network showed no significant change in mean angle measurements neither within 4 days of cultivation (from day 7 to day 11 post-seeding) nor under flow conditions, indicating that vascular network topology was maintained stable during angiogenesis. These findings stand in line with a study in yolk sacs of chicken embryos at two different developmental stages, reporting no measurable impact of hydrodynamic forces on branching angles during vascular network development. Generally, in this study, the topology of the obtained vascular network showed an anisotropic structure. Although the theoretical mean angle for both branching and branch to branch/end point angles equally distributed between 0° and 180° was expected to be 90°, it reached approximately 200 and 330 for branching and branch to branch/end point angles, respectively. Previously, published mathematical analyses claim that this anisotropic structure is based on energy optimization principles influenced mainly by circumferential stresses that dictate the morphological construction of vascular trees. The present study findings align with the theoretical optimum of a symmetrical arterial bifurcation, as predicted by optimality principle of minimum surface. Unlike the mean angle of both branching and branch to branch/end point angles, the mean vessel orientation angle indicated an isotropic structure. The mean vessel orientation angle reached approximately 0° as the expected theoretical mean angle equally distributed between −180° and 180°. These findings suggest a randomized vascular network structure that was both preserved during the angiogenesis process and unaffected by vertical direct flow conditions.

During angiogenesis, ECs form new blood vessels by sprouting or branching from pre-existing vessels. This mechanism is typically stimulated by a pro-angiogenic signal such as local gradient of VEGF. In the presence of such a signal, endothelial cells from an existing blood vessel degrade their basement membrane by secreting matrix metalloproteases (MMPs) and begin a series of events known as “tip cell selection”. This NOTCH-DLL4-dependent selection process precedes to vascular sprouting. Endothelial cells sprout within similar 3D constructs exhibited a “tip-cell” at their leading edge with visible filopodia that can be tracked manually. In order for how vascular network formation is accelerated by flow stimulation to be better understood, tip cell positions were tracked in each individual sprout during angiogenesis, in constructs cultured under direct flow versus control conditions. In a previous study applying the same manual tracking procedure, sprout directionality and speed distribution analyses in similar 3D construct cultured under static conditions indicated random endothelial sprouting directionality, with mean speed of 0.281 μm/min (16.86 μm/hr). Similar observations were made in the present study, where tip cell trajectories plotted from a shared origin point indicated an isotropic sprouting structure under control (bypass flow) conditions. Furthermore, under these conditions, endothelial tip cells resulted in a sprouting mean speed of ˜17 μm/hr. In contrast, under direct flow stimulation, tip cell trajectories displayed extended and less isotropic sprouting structures. Both mean overall tip cell sprouting distance and tip cell mean velocity under flow conditions were significantly increased as compared to those of the control tip cell. A mean sprouting speed of ˜20 μm/hr was recorded in samples subjected to sheer stimuli, while the mean sprouting speed in control samples was ˜17 μm/hr. These findings indicate that flow-induced shear stress has a significant impact on endothelial tip cell dynamics and consequently, on angiogenesis in the framework of neo-vascularization processes occurring in 3D engineered tissue. Furthermore, the anisotropic sprouting structure may be an indicator of manipulated tip cell directionality, dictated by the flow stimuli.

In an attempt to monitor and characterize cell migration and arrangement induced by wall shear stress, a cross-sectional view of a macro-channel was generated, by creating a fused 3D construct with a 1 mm diameter central hole, composed of an inner part pre-seeded with ECs and outer part pre-seeded with HNDFs (FIG. 8, C). Wall shear stress of ˜0.3 dyne/cm² (lower than the wall shear stress of ˜0.75 dyne/cm² applied in the previous experiments) was applied in order to ensure construct integrity during cultivation under flow conditions. It is considered to be the first report of long-term cross-sectional viewing of vessel dynamics under flow. During cultivation inside the flow bioreactor, HNDFs continued to proliferate massively and even penetrated and bridged the inner EC construct within 4 days of culture in the flow bioreactor. Furthermore, after 5 days of culture, HNDFs were even observed creating a smooth and rounded new inner border along the inner frame of the construct. ECs did not form the typical clusters but rather migrated to the new inner border created by the HNDFs and altered their morphology from rounded to a disc shape. Flow-induced shear stress has been shown to correlate with morphological changes in endothelial cells in vitro. This phenomenon may be indicative of a shear stress-induced cue for ECs to form an endothelial structure on the lumen created by the fibroblasts.

Tissue engineered vascular grafts (TEVG) have a broad range of clinical applications. Although these grafts have demonstrated the ability to transform into living and functioning blood vessels, the underlying mechanisms remain to be elucidated. Cultivation of a TEVG before transplantation requires dynamic culture conditions that mimic the physiological mechanical stimulations as in a matured blood vessel. Therefore, the TEVGs are usually cultured in pulsatile flow bioreactors for both improving vessel mechanical properties and possessing a confluent, adherent and quiescent endothelium to resist thrombosis in vivo. However, none of those pulsatile flow bioreactors allows live imaging of cells cultured inside the TEVG and specifically ECs lining in the lumen. In the experimental set up, a new concept of cultivating TEVGs under physiological luminal flow conditions inside the flow bioreactor was demonstrated. 4D imaging of the early 10 hours of cultivation inside the MFV bioreactor demonstrates for the first time as far as the inventors of the present invention know the dynamic of ECs migration, proliferation and morphogenesis inside of a lumen of an implantable TEVG cultured under pulsatile flow conditions.

Some embodiments are described hereinabove with reference to flowcharts and/or block diagrams depicting methods, systems and computer program products according to various embodiments.

Features of various embodiments discussed herein may be used with other embodiments discussed herein. The foregoing description of the embodiments has been presented for the purposes of illustration and description. It is not intended to be exhaustive or limiting to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes that fall within the true spirit of the present invention.

1. A flow bioreactor device for monitoring cellular dynamics comprising:
a housing having one or a plurality of conduits to place one or a plurality of tissue samples inside each of said one or a plurality of conduits,
wherein each of said one or a plurality of conduits has an inlet for introducing a flow of a liquid in a direction of flow through that conduit,
wherein said one or a plurality of conduits are fluidically linked to an outlet for discharging the liquid, and
wherein at least a portion of the housing is transparent so as to provide a line of sight for viewing or for an optical device along an elongated axis of any of said one or a plurality of conduits, the line of sight being substantially parallel to the direction of flow through that conduit.

2. The device of claim 1, wherein the housing is formed from detachable parts.

3. The device of claim 1, wherein the housing comprises a main body and a cover.

4. The device of claim 3, wherein said one or a plurality of conduits are located in the main body.

5. The device of claim 4, wherein each of said one or a plurality of conduits includes an upright portion.

6. The device of claim 3, wherein the inlet of each of said one or a plurality of conduits is located on the main body.

7. The device of claim 3, wherein each of said one or a plurality of conduits has an opening on a top surface of the main body.

8. The device of claim 7, wherein the cover has a bottom surface designed to face the top surface of the main body.

9. The device of claim 8, further comprising one or a plurality of washers for sealing one or a plurality of links between said one or a plurality of conduits and an internal chamber within the cover.

10. The device of claim 3, wherein the cover comprises a hollow body defining an internal chamber.

11. The device of claim 10, wherein the cover comprises a transparent window which, when the cover is mounted over the main body, facilitates a line of sight through the window that is aligned with said one or a plurality of conduits.

12. The device of claim 11, wherein the window is made of glass

13. The device of claim 12, wherein the window comprises a glass disk, which is secured in position by a holder.

14. The device of claim 13, wherein an O-ring is provided to seal the window.

15. The device of claim 3, wherein the main body and the cover are secured together by a screw.

16. The device of claim 1, made from one or more materials selected from the group of materials consisting of poly-methyl methacrylate (PMMA) and poly-ether ketone (PEEK).

17. The device of claim 1, wherein the device is fluidically connected to a perfusion system.

18. The device of claim 17, wherein the perfusion system is a closed-loop system.

19. The device of claim 17, wherein the perfusion system comprises one or a plurality of pumps, each of said one or a plurality of pumps is separately fluidically connected to an inlet of one of said one or a plurality of conduits.

20. The device of claim 19, wherein said one or a plurality of pumps comprises one or a plurality of peristaltic pumps.
